(19) United States
(12) Patent Application Publication
(10) Pub. No.: US 2021/0307360 A1
(43) Pub. Date: Oct. 7, 2021

(54) PROCESS FOR IMPROVING WHEAT STRAW DEGRADATION

(71) Applicant: LESAFFRE ET COMPAGNIE, Paris (FR)

(72) Inventors: Christine JULIEN, Sepx (FR); Jean-Philippe MARDEN, Labege (FR)

(73) Assignee: LESAFFRE ET COMPAGNIE, Paris (FR)

(21) Appl. No.: 16/972,216

(22) PCT Filed: Jun. 4, 2019
(86) PCT No.: PCT/FR2019/051325

§ 371 Date: Dec. 4, 2020

(30) Foreign Application Priority Data
Jun. 5, 2018 (FR) 18 54868

(51) Int. Cl. A23K 10/14; A23K 50/10; A23K 10/32

(52) U.S. Cl. CPC A23K 10/14; C12N 9/244; A23K 10/32; A23K 50/10

(57) ABSTRACT

A process for improving wheat straw degradation in a ruminant, including the following steps: a) providing the wheat straw, b) chemically pretreating the provided straw, c) feeding the ruminant with the chemically pretreated wheat straw, wherein the process is characterized in that it also includes a step d) of directly administering an enzymatic rumen additive to the ruminant, i.e. the enzymatic rumen additive is not used as a pretreatment on the wheat straw. Also, the direct use of an enzymatic rumen additive in a ruminant before, during or after feeding the ruminant with wheat straw having undergone a chemical pretreatment, i.e. the enzymatic additive is not used as a pretreatment on the wheat straw.

### FIELD OF THE INVENTION

The present invention relates to the agricultural field of animal nutrition and concerns a process for improving fodder degradation.

### TECHNOLOGICAL BACKGROUND

Wheat straw is an inexpensive resource that could help strengthen the fodder and feed autonomy of certain farms. Moreover, in some geographical areas, fodder represents a very important qualitative resource that could advantageously be valorized in animal nutrition.

However, the use of wheat straw is limited in high-producing ruminants such as cattle in particular. In fact, despite its richness in fiber, wheat straw has a low nutritional value due to poor digestion by ruminants. Indeed, from a chemical point of view, the presence of bonds between the constituents of the plant wall leads to a more or less strong resistance to the attack of the ruminal digestive microbiota. This resistance is reflected by low fodder degradation in the animal.

Thus, in order to improve the degradation of fodder, and in particular of wheat straw, research has been conducted for decades. It has been shown that the use of chemical or enzymatic pretreatments can improve degradation. It has also been shown that wheat straw degradation can be improved by the combined use of chemical and enzymatic pretreatments, said pretreatments even having a synergistic effect.

The publication by Y. Wang et al (J. Anim. Sci., 2004) presents for example the effects of an alkaline pretreatment on the efficacy of exogenous enzymes to increase wheat straw digestibility. In this publication, wheat straw is pretreated with an alkaline solution and then with an enzyme solution by spraying. The results thus show that the combination of alkaline chemical pretreatment and enzymatic treatment improves wheat straw digestibility. The enzymatic treatment employed is applied to the wheat straw prior to animal feeding in order provide a sufficient incubation time and allow an effect on digestibility. The enzymatic treatment can therefore be described as a pretreatment in the sense that it is a preliminary treatment of the wheat straw so that, once the incubation time has been respected, the wheat straw can be used later in animal feed and digestion is improved.

However, the use of enzymes in pretreatment is not efficient and has several disadvantages. Indeed, within a farm, it is time-consuming for a farmer to spray an entire stock of wheat straw. Moreover, an application time must be respected and the treatment must be applied homogeneously, which implies that the wheat straw is in a relatively free form or in bulk. Finally, the cost of enzymes is relatively high, which limits the possibility of pretreatment on a farm scale, especially when combined with chemical pretreatment which already represents an additional cost. Consequently, the combination of a chemical and enzymatic pretreatment, while the latter is effective in improving wheat straw digestibility, does not allow for widescale applications on farms due to its high implementation cost. This point is moreover raised by the publication of Y. Wang et al in the “Implication” section which explicitly mentions that the combination of an alkaline and enzymatic treatment is not economically viable.

There is thus a need to have processes for improving wheat straw degradation, said processes being inexpensive and allowing implementation on a farm scale. The valorization of wheat straw thus makes it possible to improve the fodder and overall autonomy of the farm.

It is therefore to the Inventors' credit to have developed a process that responds to all or part of the problems of prior art. The Inventors surprisingly discovered that when the enzyme additive is not used in pretreatment on the wheat straw, but rather is administered directly to the ruminant (in particular orally), before, during or after administration of the chemically pretreated wheat straw, then the effective improvement on wheat straw degradation is maintained, or even a synergistic effect appears with the chemical treatment and the enzymatic treatment.

This direct administration, in particular orally, is quite advantageous because it is simply a matter of chemically pretreating the wheat straw, and the enzyme additive can be administered before, during or after ingestion of the chemically pretreated wheat straw.

There was no suggestion that direct administration of the enzyme additive would allow a positive action on the ruminal degradation of chemically pretreated wheat straw as effective as when said enzyme additive was previously sprayed on chemically pretreated wheat straw.

Such an administration is of great interest because it allows, beyond the time savings and the simplicity of the process, the use of less enzyme additive than when the latter is sprayed on wheat straw.

### SUMMARY OF THE INVENTION

A first object of the invention relates to a process for improving ruminal wheat straw degradation in a ruminant comprising the following steps:
a) provision of wheat straw,
b) chemical pretreatment of said provided straw,
c) feeding the ruminant with the wheat straw chemically pretreated in step b),
   said process being characterized in that it further comprises a step d) of direct administration to the ruminant of a ruminal enzyme additive, which means that said enzyme additive is not used in pretreatment on the straw.

A second object of the invention relates to the direct use in a ruminant of a ruminal enzyme additive, before, during or after feeding said ruminant with wheat straw having undergone a chemical pretreatment.

“Direct” use in a ruminant means that the enzyme additive is administered directly to the ruminant, in particular orally, without having to perform a pretreatment of the wheat straw with the enzyme additive.

### DETAILED DESCRIPTION

A first object of the invention relates to a process for improving ruminal wheat straw degradation in a ruminant comprising the following steps:
a) provision of wheat straw,
b) chemical pretreatment of said provided straw,
c) feeding the ruminant with the wheat straw chemically pretreated in step b),
   said process being characterized in that it further comprises a step d) of direct administration to the ruminant of a ruminal enzyme additive, which means that said enzyme additive is not used in pretreatment on the wheat straw.

Degradation is defined as being the degradation of food under the action of microorganisms in the reticulorumen.

In the sense of the present invention, the term “pretreatment” refers to a treatment carried out on the wheat straw prior to administration to the animal, said treatment being carried out by bringing a chemical solution and the wheat straw into contact.

The process according to the invention thus improves wheat straw degradation when it is used to feed a ruminant.

Indeed, the process developed makes the parietal constituents of wheat straw more accessible to the degradation enzymes of rumen microorganisms and improves their degradation.

The magnitude of this degradation conditions digestibility throughout the digestive tract and, consequently, the energy value of the ruminant's diet.

Digestibility is a criterion that defines the degree to which organic matter is digested by an animal. The higher the digestibility, the better the digestion.

Moreover, by improving wheat straw degradation, the process according to the invention also better valorizes it in animal feed because currently it is under-used due to the difficulties the animals have in digesting it compared with other feeds.

### The First Step of the Process According to the Invention Therefore Consists in Providing Wheat Straw.

Wheat straw is the agricultural product represented by the part of the wheat stalk or stubble. The wheat straw provided is that conventionally used by the skilled person in animal feed and can be in various forms, both in the form of straw bales and in bulk form.

According to a particular embodiment, the straw may first undergo a mechanical treatment step whose objective is to reduce the size of the strands and to allow both better accessibility of the fibers to subsequent treatments and better degradation. According to this particular embodiment, the wheat straw may first undergo chopping, laceration or fiber removal, or even grinding.

### The Second Step of the Process According to the Invention Consists in Performing a Chemical Pretreatment of the Wheat Straw.

The chemical pretreatment allows in particular a substantial reduction in the rigidity of the plant structures, a swelling of the walls and their penetration by the electrolytes and the cellulolytic enzymes of the rumen microorganisms. The microorganisms can thus more rapidly colonize the plant particles which are then degraded more quickly and more intensely, thus improving degradation.

The chemical pretreatment implemented in the process of the invention is a pretreatment carried out by an alkaline solution as conventionally implemented by a skilled person. For example, the chemical pretreatment can be carried out by a sodium hydroxide (NaOH), soda, lime, potassium hydroxide (KOH), anhydrous ammonia (NH₃), aqueous ammonia (NH₄OH) or urea solution or a mixture of these compounds. Preferentially, the chemical pretreatment implemented in step b) is carried out with a sodium hydroxide (NaOH) solution.

In addition, the chemical pretreatment can be carried out in different ways according to the techniques known to the skilled person, whether industrial or not. For example, the chemical pretreatment can be carried out in a wet, semi-wet, semi-dry or dry process. According to a particular embodiment, when the pretreatment is carried out in a wet or semi-wet process, it can be applied by spraying or by soaking.

Depending on the way in which the treatment is carried out, the skilled person will adapt the concentration of the alkaline solution, as well as the post-treatment incubation times (see Chenost and Kayouli. 2.3. Chemical treatments. In: Roughage utilization in warm climates. Rome, 1997. FAO Study—Animal Production and Health—135. ISBN 92-5-203981-3).

For pretreatment with a soda solution in a semi-wet process, said solution can be concentrated from 1.6 to 5% and then mixed at a rate of 1 to 3 L/kg wheat straw. The wheat straw can then be distributed to the animals 24 to 48 hours after mixing.

For a pretreatment with a soda solution in a semi-dry process, the development of a machine coupled to a tractor power take-off to chop and mix the straw is necessary. The soda solution is concentrated midway between the semi-wet and dry processes, at 12% and then mixed in the machine with the straw at a rate of 0.4 L/kg straw. The action time of the soda is about 8 days. The straw can be dried in the open air.

For a pretreatment in a dry process, which is an industrial treatment process, the soda solution is more concentrated (16%). The solution is then mixed with chopped straw at a rate of about 0.3 L/kg wheat straw. The latter is then passed through a die press. The improving action of the soda is very rapid (from 20 seconds to about one minute) by virtue of the high temperature and pressure in the die.

Preferentially, the chemical pretreatment is carried out by a semi-wet process.

Once the Pretreatment Step has been Carried Out, the Process According to the Invention Comprises a Step of Feeding an Animal with the Chemically Pretreated Straw.

This feeding step is a conventional step well known to the skilled breeder. The animals are then fed with the pretreated wheat straw. Preferentially, the animals are ruminants such as cows or cattle, for example.

The administration step consists in administering the ruminal enzyme additive to the ruminant in such a way that it is found in the reticulorumen of said ruminant.

An example of administration of the enzyme additive is the oral route.

This administration step can be performed before, simultaneously with, or after the step of feeding the ruminant with the chemically pretreated wheat straw.

“Before” the feeding step means at most 24 hours before. Thus, the enzyme additive will never be administered more than 24 hours before the administration of the chemically pretreated wheat straw. Preferably, the ruminal enzyme additive will be administered from 2 to 8 hours before the administration of the chemically pretreated wheat straw.

Similarly, “after” the feeding step means at most 24 hours after. Thus, the enzyme additive will never be administered more than 24 hours after the administration of the chemically pretreated wheat straw. Preferably, the ruminal enzyme additive will be administered from 2 to 8 hours after the administration of the chemically pretreated wheat straw. Preferably, the administration step is carried out simultaneously with the feeding step.

Quite surprisingly, the Inventors have indeed observed that a step of direct administration to the ruminant, in particular orally, of a ruminal enzyme additive significantly improved the degradation, and even created a synergy on the degradation, with the chemical pretreatment of wheat straw.

Quite advantageously, and contrary to what was known up to now, the process according to the invention can be applied on a large scale throughout a farm and inexpensively since the enzyme is not used in pretreatment on the straw but is administered directly to the animal as an enzyme additive. This provides substantial time and cost savings. Indeed, the ruminal enzyme additive is administered directly to the ruminant before, during feeding or after feeding with the chemically pretreated wheat straw, and it is no longer necessary to spray it on the entire stock of wheat straw or the wheat straw to be consumed soon. In this way, the process according to the invention dispenses with the incubation times normally necessary to allow the enzyme to act when used in pretreatment of wheat straw.

The ruminal enzyme additive is a mixture of at least two enzymes. The enzymes used in the ruminal enzyme additive can be glucanases, xylanases, esterases, or carboxymethylcellulases.

According to a particular embodiment, the ruminal enzyme additive is a mixture of glucanase and xylanase, and in particular of endo-1,3(4)-beta-glucanase and endo-beta-1,4-xylanase.

The enzyme endo-1,3(4)-β-glucanase is a highly concentrated preparation of β-glucanase, obtained by submerged fermentation of a selected strain of Trichoderma reesei.

The enzyme endo-β-1,4-xylanase is a highly concentrated preparation of xylanase, obtained by submerged fermentation of a selected strain of Trichoderma reesei.

According to the EC nomenclature of enzymes (Enzyme Commission number):
the enzyme endo-1,3(4)-β-glucanase has the code EC 3.2.1.6,
the enzyme endo-β-1,4-xylanase has the code EC 3.2.1.8.

According to this embodiment, the dose of glucanase can be comprised between 200 units/g straw and 7000 units/g straw, preferably from 4000 units/g straw to 6000 units/g straw, such as for example about 4670 units/g straw.

The dose of xylanase can be comprised between 200 units/g straw and 7000 units/g straw, preferably from 4000 units/g straw to 6000 units/g straw, such as for example about 4670 units/g straw.

Enzyme units correspond to the amount of enzyme needed to:
release 1 μmole of glucose per minute at pH 4.8 and 50° C., from a barley glucan substrate for glucanase;
release 1 μmole of xylose per minute at pH 5.3 and 50° C. from a birchwood xylan substrate for xylanase.

Still according to this embodiment, the glucanase can be an endo-1,3(4)-β-glucanase and the xylanase can be an endo-β-1,4-xylanase. In this case, and advantageously, the glucanase/xylanase ratio is at least 0.5, preferably at least 0.75, and especially about 1.

The ruminal enzyme additive is in a form suitable for animal administration. For example, the ruminal enzyme additive can be in the form of capsules, bolus, powder, granules or liquid, such as for example in ampoules. Preferably, the ruminal enzyme additive is in powder or liquid form, preferably in powder form.

A capsule is a solid preparation, consisting of a hard or soft shell of variable shape and capacity.

A tablet or a bolus is a solid preparation containing a unit dose of one or more active principles (or preparation). They are obtained by compression of a constant volume of particles. Intended for livestock, they become larger and are then called a bolus.

According to a particular embodiment of the invention, when the ruminal enzyme additive is administered simultaneously with the chemically pretreated wheat straw, said ruminal enzyme additive can be in the form of a powder which will be mixed with the chemically pretreated wheat straw. The mixture is then used to feed the ruminant.

According to another embodiment of the invention, the ruminant belongs to the family Bovidae. The family of bovids (Bovidae) comprises several subfamilies, notably including the Bovinae (of which bovines are part) and Caprinae (of which sheep and goats are part).

The improvement of wheat straw degradation can be determined by techniques known to the skilled person, such as for example by measurements of dry matter loss by incubation in ruminal fluid. Details of the measurements are presented in the example section below.

The process according to the invention is particularly advantageous because it allows surprisingly a synergistic action between the ruminal enzyme additive and the chemical pretreatment of wheat straw on wheat straw degradation.

Indeed, the effect on ruminal degradation is greater than the sum of the combined effects of the chemical pretreatment and the ruminal enzyme additive alone.

No process known to date improved wheat straw degradation to a level equivalent to the improvement made by the process according to the invention.

It is known to use a chemical pretreatment, and in particular an alkaline pretreatment for the treatment of wheat straw to improve its digestibility. It is also known to use an enzymatic pretreatment to improve digestibility.

Up to the present, treatments carried out on straw, whether chemical or enzymatic, were pretreatments in the sense that they were carried out before ingestion by the animal, by pretreatment of the wheat straw itself.

Enzymatic pretreatment is known to improve straw digestibility in animals, but this type of pretreatment is hardly ever used because it is too expensive to spray or pretreat entire batches of straw for subsequent feeding to the animal. The objective of these pretreatments is to achieve better potentiation of a food source that has been little valorized to date. However, the use of enzymatic pretreatments, even though they have proved to be of interest for improving digestibility, has not been developed in the agricultural community because the necessary cost of an enzymatic pretreatment is not compensated by the gain in terms of feed for the animals.

It has already been proven in the prior art that the combination of a chemical and enzymatic pretreatment of straw can improve the digestibility of straw and even have a synergy. However, due to the costs of in situ or ex vivo enzymatic pretreatment of wheat straw, the combination of the two pretreatments has never been implemented on a farm scale and was considered up to now unsuitable by the person skilled in the art.

It is therefore to the merit of the present invention to have been able to develop a process allowing an improvement in straw degradation, said process being able to be implemented on the scale of a cattle farm without presenting additional work or harmful additional costs. On the contrary, the process according to the invention potentiates the wheat straw and thus allows animals to better digest this food which sometimes constitutes one of the main resources.

By virtue of the process according to the invention, the wheat straw is better valorized and it is simpler for the farms to tend towards feed self-sufficiency without generating substantial additional costs.

A second object of the invention relates to the direct use in a ruminant of a ruminal enzyme additive before, after or simultaneously with the feeding of said ruminant with wheat straw having first undergone an alkaline pretreatment, and preferably simultaneously with the feeding of said ruminant, which means that said enzyme additive is not used in pretreatment on the wheat straw.

Said ruminal enzyme additive is an enzyme mixture comprising at least two enzymes. The enzymes used in the ruminal enzyme additive can be glucanases, xylanases, esterases, or carboxymethylcellulases.

According to a particular embodiment, the ruminal enzyme additive is a mixture of glucanase and xylanase and in particular of endo-1,3(4)-beta-glucanase and endo-beta-1,4-xylanase.

According to this embodiment, the dose of endo-1,3(4)-β-glucanase can be comprised between 200 units/g straw and 7000 units/g straw, preferably from 4000 units/g straw to 6000 units/g straw, such as for example about 4670 units/g straw.

Similarly, the dose of endo-β-1,4-xylanase can be comprised between 200 units/g straw and 7000 units/g straw, preferably from 4000 units/g straw to 6000 units/g straw, such as for example about 4670 units/g straw.

Advantageously, the ratio of endo-1,3(4)-β-glucanase to endo-β-1,4-xylanase is at least 0.5, preferably at least 0.75, and especially about 1.

The ruminal enzyme additive is in a form suitable for animal administration. For example, the ruminal enzyme additive can be in the form of capsules, bolus, powder, granules or liquid, such as for example in ampoules. Preferably, the ruminal enzyme additive is in powder or liquid form, preferably in powder form.

The use according to the invention is particularly advantageous because it improves wheat straw degradation by animals. Moreover, simultaneous use creates a synergistic effect on wheat straw degradation.

The invention will be better understood with the help of the following examples which are purely illustrative and in no way provide a limitation.

### FIGURES

FIG. 1: Comparison of the effect of an aqueous (H₂O) or alkaline (NaOH 5%) pretreatment alone, of the use of a ruminal enzyme additive alone (represented hereinafter by (GP+XP)×10) or the combination of an alkaline pretreatment and a ruminal enzyme additive (represented hereinafter by (NaOH 5%+(GP+XP)×10)) on wheat straw dry matter degradation (in percentage % disappearance/degradation of the wheat straw dry matter).

The two dotted lines represent respectively the value of the disappearance/degradation of the dry matter of wheat straw pretreated with soda (NaOH 5%) (upper line) and not pretreated (control (C)) (bottom line).

### EXAMPLES

### Materials and Methods

1. Preparation of Fermentation Modules

Wheat straw is crushed on a 2 cm mesh (Electra) and then the fine particles are removed using a 1.18 mm sieve (Penn State Separator).

Fractions (3 g) are then weighed in triplicate and placed in bottles equipped with a pressure measurement module (Ankom Technology).

2. Pretreatment

A pretreatment is applied by a semi-wet process for 24 hours on the straw before incubation with rumen fluid. Two types of pretreatment are applied. A water pretreatment (control) and a chemical pretreatment.

The water pretreatment is a negative control. Indeed, the pretreatment is done by a solution of soda in water. Both (i) a negative control pretreated with water only and (ii) a negative control without pretreatment are therefore needed to then dissociate the effect of water pretreatment and that of soda, and thus to be able to conclude specifically about the effect of soda as pretreatment.

The chemical pretreatment is applied by bringing the wheat straw into contact with a 5% sodium hydroxide (NaOH) solution. 9 mL of solution is used for 3 g of wheat straw.

3. Additive

In order to recreate the in vivo conditions of co-administration of wheat straw and the enzyme additive according to the process of the invention, the additive is added to the modules at the time of incubation with ruminal fluid.

The ruminal enzyme additive is a mixture of two enzymes in a 1:1 ratio:
an endo-1,3(4)-beta-glucanase, hereinafter GP, having an activity of 70000 units/g and
an endo beta 1-4 xylanase, hereinafter XP, having an activity of 70000 units/g.

Enzyme units correspond to the amount of enzyme needed to:
release 1 μmole of glucose per minute at pH 4.8 and 50° C. from a barley glucan substrate for glucanase;
release 1 μmole of xylose per minute at pH 5.3 and 50° C. from a birchwood xylan substrate for xylanase.

Ten doses of the enzyme mixture ((GP+XP)×10) corresponds to 0.4 g of the enzyme mixture for 3 g of straw, i.e. 0.2 g of GP and 0.2 g of XP for 3 g of straw, i.e. 4670 units (rounded to the nearest ten) of GP and XP for 1 g of straw.

4. Obtaining Ruminal Fluid for Fermentation

Ruminal contents are collected about 2 hours after feeding from dry dairy cows, carrying a ruminal cannula and fed a ration based on wheat straw, corn silage and a mineral and vitamin supplement.

After collection, the ruminal contents are filtered through a metal sieve with a 1.6 mm mesh size in order to obtain the ruminal fluid.

The ruminal fluid is then mixed with a pH=7 buffer solution (ratio 1:1) to form the incubation medium. The incubation medium is bubbled with oxygen-free CO₂

5. Preparation of the Fermentation Modules

Several modules are prepared and distributed within 4 tests. The composition of the different modules and their distribution within the tests are presented below.

### Test 1: Pretreated Wheat Straw Only

6 modules containing straw chemically pretreated with an alkaline solution according to the protocol of point 2. above;
3 modules containing straw pretreated according to the same protocol as point 2. but replacing the alkaline solution with water;
3 control modules containing only straw, hereinafter C.

### Test 2: Non-Pretreated Wheat Straw and Ruminal Enzyme Additive (See Point 3. Above).

3 modules containing non-pretreated straw and a 10× concentrated ruminal additive ((GP+XP)×10);
3 control modules containing only straw, hereinafter C.

### Test 3: Duplicate of Test 2 to Validate and Confirm the Results.

### Test 4: Pretreated Wheat Straw (See Point 2.) and Ruminal Enzyme Additive (See Point 3.)

6 modules containing straw chemically pretreated with an alkaline solution according to the protocol of point 2. and a 10× concentrated ruminal enzyme additive ((GP+XP)×10);
3 control modules containing only straw, hereinafter C.

For each of the tests, the non-pretreated straw corresponds to straw pretreated according to the same protocol as in point 2. but replacing the alkaline solution with water.

For each test, 3 blank modules are made. They contain only the incubation medium as described in point 4. of the present example.

The incubation bottles receive 200 mL of incubation medium each and are then saturated with oxygen-free CO₂ The amount of pretreated or non-pretreated straw within each module is 3 grams.

6. Measurement of the Degraded Dry Matter.

The bottles are incubated at 39° C. for 96 hours to allow fermentation.

After 96 h of incubation, the modules are placed in ice to stop fermentation. The amount of undegraded dry matter is then measured by filtering and drying the incubation residue at 60° C. for 4 days (hereinafter called residue).

The incubated substrate corresponds to the pretreated or non-pretreated wheat straw.

The disappearance of dry matter (in percentage %) is calculated according to the following formula:

[incubated substrate−(incubated substrate residue−control module residue)]/incubated substrate×100

Whether for the incubated substrate, the residues of the incubated substrate or of the control module, it is the average of the results obtained for the modules of the same treatment.

7. Statistical Analysis

The dry matter degradation results (in percentages %) were subjected to an analysis of variance using the linear general univariate model of the statistical data processing software SPSS® (IBM®, SPSS®, version 22). The model incorporates the effects of the covariate and the products, the covariate being the dry matter degradation of the controls (in percentage %). A log 10 transformation of the values expressed in percentage was performed for the analysis.

Results are considered insignificant when p>0.05.

The different letters a, b, c, d used in the FIGURE indicate whether values are significantly different or not. Thus, the results are not significantly different when the same letter is reported (p>0.05).

### Results

### Effect of the Use of an Alkaline Pretreatment and a Ruminal Enzyme Additive

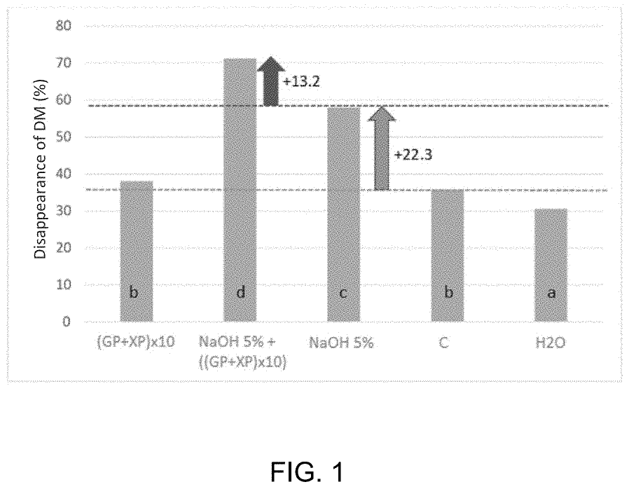

FIG. 1 highlights a significant effect on ruminal dry matter degradation of the use of a ruminal enzyme additive on wheat straw pretreated with soda (NaOH 5%+(GP+XP)×10).

The results show that a pretreatment with soda (5%) improves ruminal straw degradation compared with (i) no pretreatment and (ii) a pretreatment with water. These results thus confirm the direct effect of the soda used in pretreatment on the ruminal straw degradability.

The pretreatment with soda improves ruminal dry matter degradation by 22.3 points compared with non-pretreated wheat straw (control).

No significant increase in dry matter consumption is however observed on the non-pretreated straw in the presence of an addition of the ruminal enzyme additive ((GP+XP)×10) compared with non-pretreated wheat straw (control).

In contrast, quite surprisingly, the addition of a ruminal enzyme additive on straw chemically pretreated with soda makes it possible to highlight a synergistic effect on ruminal dry matter degradation.

The synergistic effect is understood as the fact that the ruminal degradation in the animal of chemically pretreated wheat straw in the presence of a ruminal enzyme additive is higher than the ruminal degradation obtained by a chemical pretreatment alone, by the administration of the enzyme additive alone or by the two combined but working independently.

Indeed, the addition of the ruminal enzyme additive ((GP+XP)×10) on wheat straw pretreated with soda increases said consumption by 13.2 points respectively.

The process according to the invention is therefore particularly advantageous in that it significantly improves ruminal wheat straw degradation when the wheat straw is chemically pretreated.

1-18. (canceled)

19. A process for improving ruminal wheat straw degradation in a ruminant comprising the following steps:
a) provision of wheat straw,
b) chemical pretreatment of said provided straw,
c) feeding the ruminant with the wheat straw chemically pretreated in step b),
wherein said process further comprises a step d) of direct administration to the ruminant of a ruminal enzyme additive, which means that said enzyme additive is not used in pretreatment on the wheat straw.

20. The process as claimed in claim 19, wherein the chemical pretreatment of step b) is an alkaline pretreatment.

21. The process as claimed in claim 20, wherein the alkaline pretreatment is a pretreatment carried out with a sodium hydroxide, soda, lime, potassium hydroxide, anhydrous ammonia, aqueous ammonia or urea solution or a mixture of these compounds, preferably the alkaline treatment is carried out with a sodium hydroxide solution.

22. The process as claimed in claim 19, wherein the chemical pretreatment is carried out by a wet, semi-wet, dry or semi-dry process, and preferably by a semi-wet process.

23. The process as claimed in claim 19, wherein step d) of direct administration of the ruminal enzyme additive is an oral administration.

24. The process as claimed in claim 19, wherein step d) of administration of the ruminal enzyme additive is carried out before, after or simultaneously with step c) of feeding the ruminant with the pretreated wheat straw, preferably simultaneously with step c).

25. The process as claimed in claim 19, wherein the ruminal enzyme additive is an enzyme mixture comprising at least two enzymes selected from glucanases, xylanases, esterases, or carboxymethylcellulases.

26. The process as claimed in claim 25, wherein the enzyme additive is an enzyme mixture comprising a glucanase and a xylanase, preferably an endo-1,3(4)-β-glucanase and an endo-β-1,4-xylanase.

27. The process as claimed in claim 26, wherein endo-1,3(4)-β-glucanase is present in a dose comprised between 200 units/g straw and 7000 units/g straw, preferably from 4000 units/g straw to 6000 units/g straw, and even more preferentially 4670 units/g straw.

28. The process as claimed in claim 26, wherein endo-β-1,4-xylanase is present in a dose comprised between 200 units/g straw and 7000 units/g straw, preferably from 4000 units/g straw to 6000 units/g straw, and even more preferentially 4670 units/g straw.

29. The process as claimed in claim 26, wherein endo-1,3(4)-β-glucanase and endo-β-1,4-xylanase are present in a ratio of at least 0.5, preferably in a ratio of at least 0.75 and even more preferably in a ratio of 1:1 in the enzyme mixture.

30. The process as claimed in claim 19, wherein the ruminal enzyme additive is in powder or liquid form, preferably in powder form.

31. The process as claimed in claim 19, wherein the ruminant belongs to the family Bovidae.

32. A process for improving ruminal wheat straw degradation in a ruminant, comprising administering to a ruminant a ruminal enzyme additive before, after or simultaneously with the feeding of said ruminant with wheat straw having first undergone an alkaline pretreatment, preferably simultaneously with the feeding of said ruminant, which means that said enzyme additive is not used in pretreatment on the wheat straw.

33. The process as claimed in claim 32, wherein said ruminal enzyme additive is an enzyme mixture comprising at least two enzymes selected from glucanases, xylanases, esterases, or carboxymethylcellulases, preferably a glucanase and a xylanase and even more preferably an endo-1,3(4)-β-glucanase and an endo-β-1,4-xylanase.

34. The process as claimed in claim 33, wherein the endo-1,3(4)-β-glucanase is present in a dose comprised between 200 units/g straw and 7000 units/g straw, preferably from 4000 units/g straw to 6000 units/g straw, and even more preferentially 4670 units/g straw.

35. The process as claimed in claim 33, wherein the endo-β-1,4-xylanase is present in a dose comprised between 200 units/g straw and 7000 units/g straw, preferably from 4000 units/g straw to 6000 units/g straw, and even more preferentially 4670 units/g straw.

36. The process as claimed in claim 33, wherein the endo-1,3(4)-β-glucanase and endo-β-1,4-xylanase are present in a ratio of at least 0.5, preferably in a ratio of at least 0.75 and even more preferably in a ratio of 1:1 in the enzyme mixture.
